(19) United States
(12) Patent Application Publication
(10) Pub. No.: US 2026/0014143 A1
(43) Pub. Date: Jan. 15, 2026

(54) Prostaglandin E synthase 3 (PTGES3) Inhibiting Compounds

(71) Applicant: The Regents of the University of California, Oakland, CA (US)

(72) Inventors: James E. Melnyk, San Francisco, CA (US); Luke A. Gilbert, San Francisco, CA (US); Kevan M. Shokat, San Francisco, CA (US); Felix Y. Feng, San Francisco, CA (US); Haolong Li, San Francisco, CA (US)

(21) Appl. No.: 18/992,423

(22) PCT Filed: Jul. 19, 2023
(86) PCT No.: PCT/US2023/028150

§ 371 Date: Jan. 8, 2025

Related U.S. Application Data
(60) Provisional application No. 63/369,051, filed on Jul. 21, 2022.

(51) Int. Cl. A61K 31/496; A61K 31/4025; A61P 35/00; C07D 207/14; C07D 295/26; C07D 403/06; C12N 9/99

(52) U.S. Cl. CPC A61K 31/496; A61K 31/4025; A61P 35/00; C07D 207/14; C07D 295/26; C07D 403/06; C12N 9/99

(57) ABSTRACT

Provided are compounds for inhibiting prostaglandin E synthase 3 (PTGES3) along with methods of treating prostate cancer by administering such compounds to a subject. The PTGES3 inhibitors has a moiety that can form a covalent bond with a thiol group, for example, of a cysteine residue of PTGES3. By forming such a bond, the inhibitor can block a site on PTGES3 that is involved with the interaction between PTGES3 and androgen receptor (AR). Since the progression of prostate cancer can depend on AR activity, blocking the interaction of PTGES3 and AR can be used to treat prostate cancer.

### CROSS REFERENCE TO RELATED APPLICATION

This application claims the benefit of U.S. Provisional Patent Application No. 63/369,051, filed Jul. 21, 2022, which application is incorporated herein by reference in its entirety.

### GOVERNMENT RIGHTS

This invention was made with government support under grant numbers 1F32CA236347-01, 1R01CA221969-01, 1R01CA244550, K99/R00CA204602, DP2 CA239597, and P50CA186786 awarded by the National Institutes of Health (NIH), along with grant number 1R01CA227025 co-awarded by the NIH and the National Cancer Institute (NCI). The government of the United States has certain rights in the invention.

### INTRODUCTION

Prostate cancer is the second most diagnosed cancer in men and the sixth most common cause of cancer death for men (Baade et al., Molecular Nutrition & Food Research, 2009, 53, 2, 171, doi:10.1002/mnfr.200700511). Ferley et al. estimated that prostate cancer has a mortality rate of about 9.5% and would result in about 92,000 cancer deaths per year in Europe (European Journal of Cancer, 2013, 49, 6, 1374, doi: 10.1016/j.ejca.2012.12.027).

Common treatments for prostate cancer include androgen deprivation and the suppression of androgen receptor (AR) signaling through an AR antagonist or antiandrogens (Helsen et al., Endrocrine-Related Cancer, 2014, 21, 4, doi:10.1530/ERC-13-0545). The androgen receptor is a nuclear receptor that is activated by the binding of androgenic hormones, such as testosterone and dihydrotestosterone. The FOXAT and HOXB13 genes are key drivers of prostate cancer (Pomerantz et al., Nature Genetics, 2015, 47, 1346, doi:10.1038/ng.3419 and Parolia et al., Nature, 2019, 571, 413, doi:10.1038/s41586-019-1347-4).

In addition, Gilbert et al. (WO 2021/154974) reported that prostaglandin E synthase 3 (PTGES3) inhibitors could be useful for the treatment of prostate cancer by modulating AR levels.

### SUMMARY

Provided are compounds for inhibiting prostaglandin E synthase 3 (PTGES3) along with methods of treating prostate cancer by administering such compounds to a subject. The PTGES3 inhibitors has a moiety that can form a covalent bond with a thiol group, for example, of a cysteine residue of PTGES3. By forming such a bond, the inhibitor can block a site on PTGES3 that is involved with the interaction between PTGES3 and androgen receptor (AR). Since the progression of prostate cancer can depend on AR activity, blocking the interaction of PTGES3 and AR can be used to treat prostate cancer.

### BRIEF DESCRIPTION OF THE DRAWINGS

FIG. 1A shows a surface cysteine (Cys76) is proximal to the catalytic tyrosine residue (Tyr9) and the tryptophan residue (Trp106) at the co-chaperone binding site (PDB: 1EJF).

FIG. 1B shows LNCaP (TET-ON PTGES3) expressing flag-tagged wildtype or C76W were treated DMSO or 100 ng/ml doxycycline. Co-IP assays were performed using flag antibody. AR and HSP90 levels were detected by western blotting. The indole side chain of tryptophan serves as a proxy for small molecule binding, and the tryptophan mutant of a protein can mimic behavior observed when a small molecule is bound at the site of the tryptophan mutation.

FIG. 1C shows evaluation of the top six fragments for labeling of recombinant CysLite PTGES3 at high concentrations of BME competitor (n=3 as independent replicates; Mean f SEM).

FIG. 1D shows the top fragments were evaluated by differential scanning fluorimetry for stabilization of PTGES3. Fragment 994364 increases the thermal melt temperature of PTGES3. The plot is of the average values of stabilization.

FIG. 1E shows the non-disulfide version of the top hit 1 was synthesized as a mixture of the trans-enantiomers, Compound 2.

FIG. 1F shows LNCaP cells were treated with DMSO (grey) or 100 μM Compound 2 (blue) for 48 h. Nuclear AR and TBP levels were detected by western blotting. Each western blot experiment was performed twice to determine reproducibility.

FIG. 1G shows LNCaP cells were treated with DMSO or 100 μM Compound 2 for 48 h. RNA was collected, indicated genes mRNA levels over 18s rRNA were measured by real-time PCR (n=3 as biological replicates; Mean±SEM).

FIG. 1H shows Nuclight red labeled LNCaP or PC3 cells were treated with a dose response of 9. Normalized to DMSO and analyzed by IncuCyte system (n=3 as biological replicates; Mean±SEM).

FIG. 1I shows Nuclight red labeled P3C cells were treated with DMSO (grey) or 100 μM Compound 2 (blue). Fold change of cell count normalized over day 0 was analyzed by IncuCyte system (n=3 as biological replicates; Mean SEM). Unpaired two-tailed t-test was used to determine statistical significance (ns=no significant difference; *** P<0.001).

FIG. 2A shows fragment library consisting of 1800+ disulfide-linked monophores were screened against untagged, recombinant CysLite PTGES3. A hit threshold of > than 65% modification (blue line) was selected yielding a hit rate of 0.65% (red dots).

FIG. 2B shows a top fragment hit for further evaluation for binding to CysLite PTGES3.

FIG. 2C shows EC₅₀ values for labeling of recombinant CysLite PTGES3 in the presence of 100 μM BME determined for the top six fragment hits (N.D.=Not determinable).

FIG. 2D shows the top hit, 994364, was re-synthesized as a mixture of the trans-enantiomers (1).

FIG. 2E shows Top hit re-synthesized as 1 and confirmed by DSF to stabilize CysLite PTGES3 (n=3 as independent replicates; Mean±SEM).

FIG. 3A shows far Western blot analysis of AR-PTGES3 interaction. Flag-AR was loaded on a gel, transferred to membrane, and renatured. The membranes were incubated with recombinant CysLite PTGES3 protein or recombinant CysLite PTGES3 protein labeled with 1. The interaction was detected with PTGES3 antibody. The blot shows 1 disrupted the PTGS3-AR interaction. Each blot experiment was performed twice to determine reproducibility.

FIG. 3B shows Recombinant AR protein, IgG, CysLite PTGES3, ARE DNA, and Compound 1 were mixed as indicated in a custom reaction buffer (DHT+). The reactions were then loaded on a DNA retardation gel. ARE DNA bands were detected by SYBR gold. When comparing lanes 3 with 4, addition of Compound 1 increases the free ARE levels by inhibiting PTGES-AR-ARE binding. Experiment was performed twice to determine reproducibility.

### DETAILED DESCRIPTION

Provided are compounds for inhibiting prostaglandin E synthase 3 (PTGES3) along with methods of treating prostate cancer by administering such compounds to a subject. The PTGES3 inhibitors has a moiety that can form a covalent bond with a thiol group, for example, of a cysteine residue of PTGES3. By forming such a bond, the inhibitor can block a site on PTGES3 that is involved with the interaction between PTGES3 and androgen receptor (AR). Since the progression of prostate cancer can depend on AR activity, blocking the interaction of PTGES3 and AR can be used to treat prostate cancer.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases the alkyl group has 1 to 24 carbon atoms, e.g. 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g. O, S, N) as a ring atom and that is not aromatic (i.e. distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e. none of the ring atoms are heteroatoms (e.g. O, S, N). In some cases the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g. O, S, N). Exemplary heteroaryl groups include those obtained from removing a hydrogen atom from pyridine, pyrimidine, furan, thiophene, or benzothiophene.

The term “substituted” refers the removal of one or more hydrogens from an atom (e.g. from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some cases, the substitutions can themselves be further substituted with one or more groups. For example, the group —C₆H₄CH₂CH₃ can be considered as substituted aryl, i.e. an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein. In some cases, the substituents are not substituted with any other groups.

Diradical groups are also described herein, i.e. in contrast to the monoradical groups such as alkyl and aryl described above. The term “alkylene” refers to the diradical version of an alkyl group, i.e. an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e. a CHCH₃ fragment where the first atom has two single bonds to other two different groups). The term “arylene” refers to the diradical version of an aryl group, e.g. 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is any non-hydrogen group, e.g. alkyl, alkenyl, alkynyl, or substituted versions thereof. For example, the acetyl group has formula —C(O)CH₃. “Acylene” refers to a diradical group of formula —C(O)R—, e.g. wherein R is alkylene or cycloalkylene. “Carbonyl” refers to a diradical group of formula —C(O)—.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein R^{X} and R^{Y} are each independently H or a non-hydrogen substituent. Exemplary non-hydrogen substituents include alkyl groups (e.g. methyl, ethyl, and isopropyl).

“Carbonyl” refers to a diradical group of formula —C(O)—.

“Carboxy” is used interchangeably with carboxyl and carboxylate to refer to the —CO₂H group and salts thereof.

“Ether” refers to a diradical group of formula —O—. For instance, if the ether group is connected to an alkyl group, then the overall group is an alkoxy group (e.g. —OCH₃ or methoxy). If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

“Sulfonyl” and “sulfone” refers to a group of formula —SO₂—.

“Sulfonylamine” refer to a group of formula —SO₂-(amino). For instance, the sulfonylamine group can have the formula —SO₂NH₂.

Unless otherwise specified, reference to an atom is meant to include all isotopes of that atom. For example, reference to H includes ¹H, ²H (i.e. D or deuterium) and ³H (i.e. tritium), and reference to C is includes both ¹²C and all other isotopes of carbon (e.g. ¹³C). Unless specified otherwise, groups include all possible stereoisomers.

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., cancer, etc.) present in the subject.

### Compounds

Provided are compounds for inhibiting PTGES3. In some cases, the compound has formula (I):

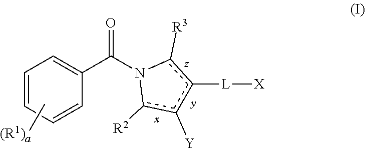

wherein:
each R¹, R² and R³ is independently selected from H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof;
L is a linking group or absent;
X is a thiol covalently bonding moiety;
Y is —(CH₂)ₙR⁵, wherein n is 0 or 1, wherein R⁵ is selected from aryl, substituted aryl, heteroaryl, substituted heteroaryl, cycloalkyl, and substituted cycloalkyl;
a is an integer ranging from 0 to 5; and
the covalent bonds designated “x”, “y” and “z” are each independently a single bond or a double bond provided that if bond “y” is a double bond then bonds “x” and “z” are single bonds.

In some cases, each R¹ is independently selected from halo, hydroxy, sulfonylamine, alkyl, alkoxy, cyano, and nitro. In some cases, a ranges from 1 to 5 and at least one R¹ is selected from halo, hydroxy, sulfonylamine

For instance, in some cases at least one R¹ group is present, i.e. “a” ranges from 1 to 5. In some embodiments, at least R¹ is selected from halo, hydroxy, and sulfonylamine. In some cases a is 2, a is 3, a is 4, or a is 5. In some cases, there is at least one R¹ group present that is sulfonylamine, e.g. of formula —SO₂NH₂. In some cases there is at least one R¹ group present that is a sulfonylamine and at least one R¹ group that is a halogen, e.g. Cl or F. In some embodiments there is at least one R¹ group present that is a sulfonylamine, at one least R¹ groups is a halogen, and at least one R¹ group is hydroxy.

As described above, X is a thiol covalently bonding moiety. As such, the X moiety is configured to form a covalent bond with a thiol group, e.g. a thiol group of a cysteine group of the PGES3 protein. As used herein, the “thiol group” refers to a group of formula —SH and salts thereof, e.g. —S⁻Na⁺.

In some instances, the formation of the covalent bond between X and a thiol group involves a nucleophilic reaction, e.g. wherein the thiol group acts as a nucleophile and the X group acts as an electrophile. Thus, in some cases the thiol covalently bonding moiety (i.e. X) is an electrophilic group. The location on X where the thiol nucleophile bonds to X is referred to herein as the electrophilic site. In some cases, X further includes an electron-withdrawing group that withdraws electron density from the electrophilic site, thereby increasing its electrophilicity. In some embodiments X includes a r conjugation group that is 7r conjugated to the electrophilic site, thereby making it more electrophilic. In some instances the thiol covalently bonding moiety is a Michael acceptor.

In some embodiments, the thiol covalently bonding moiety is selected from the group consisting of a vinyl sulfone, a maleimide, a α-halocarbonyl, an acrylamide, and a β-haloethylamine, an iodoacetamide (or α-haloamide), an epoxide, or an azirdine. For instance, the thiol covalently bonding moiety can be a vinyl sulfone group, e.g. a vinyl sulfonamide. Exemplary thiol covalently bonding moieties are described by Lopez-Jaramillo et al (“Vinyl Sulfone: A Multi-Purpose Function in Proteomics”, doi:10.5772/29682), Brosnan et al (“Modification of Polypeptide Materials by Thiol-X Chemistry”, Polymer, 2014, 55, 5511, doi:10.1016/j.polymer.2014.08.067), and Craven et al (“Vinyl sulfonamide synthesis for irreversible tethering via a novel α-selenoether protection strategy”, MedChemComm, 2019, 10, 158, doi:10.1039/c8md00566d).

As described above, L is a linking group or L is absent. In some embodiments, L is absent and the thiol covalently bonding moiety (X) is directly bonded to the nitrogen-containing 5-membered ring. In other cases, L is a linking group that is present. As used herein, the linking group L refers to any diradical group of 30 atoms or less that is covalently bonded to both the 5-membered nitrogen-containing ring and the X group. In some embodiments, L is alkylene, alkenylene, alkynylene, cycloalkylene, heterocyclylene, arylene, heteroarylene, amino, acylene, and substituted versions thereof. For instance, L can be an acylene group which has the formula —C(O)R′—, wherein R′ is any diradical group, e.g. amino, cycloalkylene, and heterocyclylene. In some cases, L has the formula —C(O)—.

In some instances, R² is H. In some instances R³ is H. In some embodiments the covalent bond designated “z” is a single bond, whereas in other cases the covalent bond designated “z” is a double bond.

In some cases, the compound has formula (Ia):

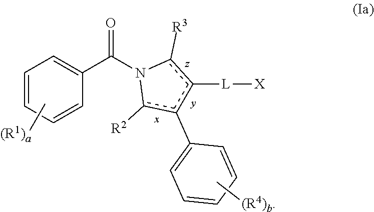

As described above in reference to formula (I), Y is —(CH₂)ₙR⁵, wherein n is 0 or 1, wherein R⁵ is selected from aryl, substituted aryl, cycloalkyl, and substituted cycloalkyl. In some cases, n is 1 and R⁵ is aryl or substituted aryl. In some cases, n is 0 and R⁵ is aryl or substituted aryl.

In some instances, the compound has formula (II):

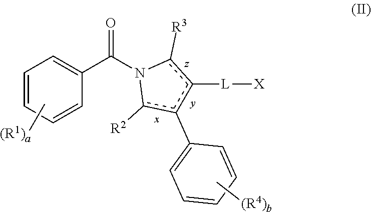

wherein:
each R⁴ is independently selected from alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and
b is an integer ranging from 0 to 5.

In some cases, the compound has formula (IIa):

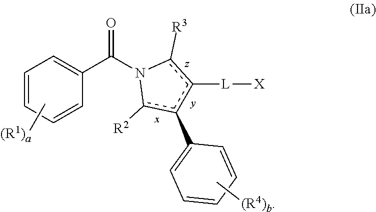

In some instances, the compound has formula (III):

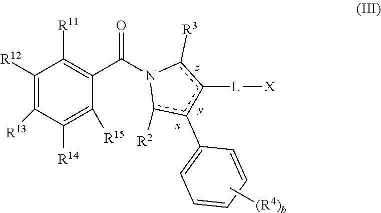

wherein:
R¹¹, R¹², R¹³, R¹⁴, and R¹⁵ are each independently selected from H, halo, hydroxy, and sulfonylamine.

In some embodiments, the compound has formula (IIIa):

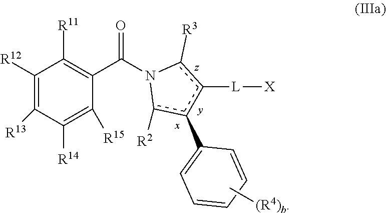

In some embodiments of formula (III) or (IIIa), R¹¹ is H. In some cases, R¹² is halo, e.g. chloro or fluoro. In some cases, R¹³ is hydroxy. In some embodiments, R¹³ is sulfonylamine. In some cases R¹⁴ is halo, e.g. chloro or fluoro. In some cases, R¹⁵ is H. In some embodiments, R² is H. In some cases, R³ is H. In some instances, the covalent bond designated “z” is a single bond.

In some cases the PTGES3 inhibiting compound has a structure selected from the group consisting of:

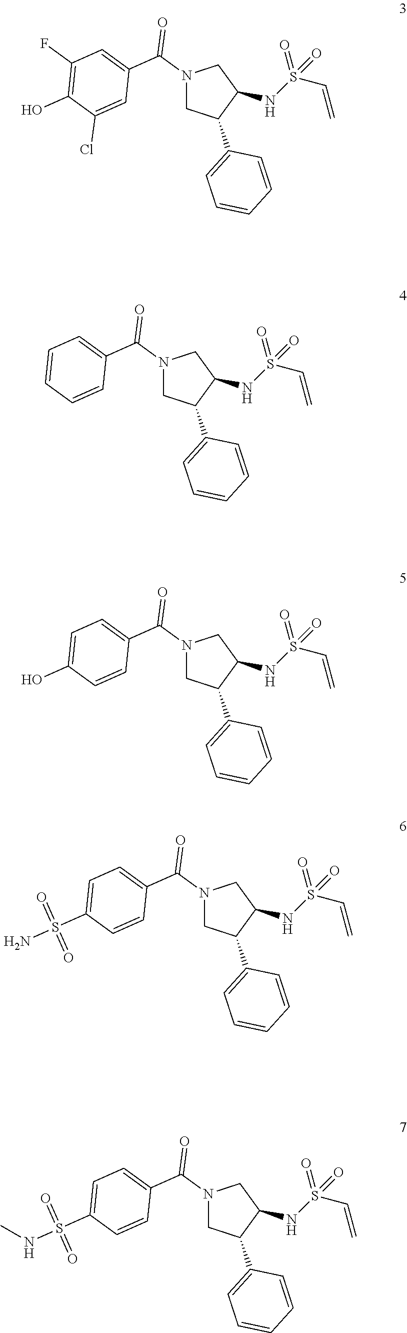

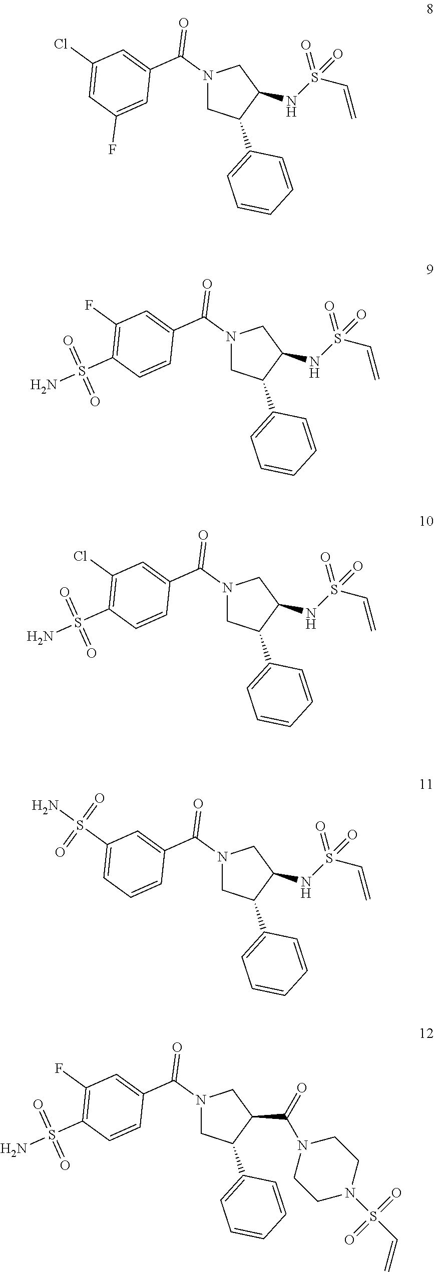 [SMILES: C=CS(=O)(=O)N[C@@H]1CN(C(=O)c2cc(F)cc(Cl)c2)C[C@H]1c1ccccc1 ; C=CS(=O)(=O)N[C@@H]1CN(C(=O)c2ccc(S(N)(=O)=O)c(F)c2)C[C@H]1c1ccccc1 ; C=CS(=O)(=O)N[C@@H]1CN(C(=O)c2ccc(S(N)(=O)=O)c(Cl)c2)C[C@H]1c1ccccc1 ; C=CS(=O)(=O)N[C@@H]1CN(C(=O)c2cccc(S(N)(=O)=O)c2)C[C@H]1c1ccccc1 ; C=CS(=O)(=O)N1CCN(C(=O)[C@@H]2CN(C(=O)c3ccc(S(N)(=O)=O)c(F)c3)C[C@H]2c2ccccc2)CC1]

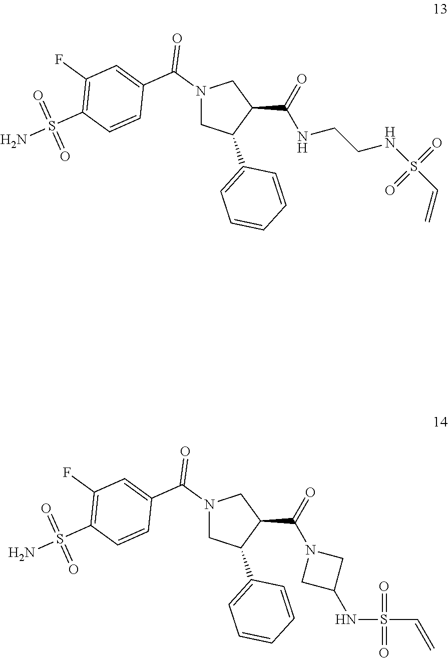 [SMILES: C=CS(=O)(=O)NCCNC(=O)[C@@H]1CN(C(=O)c2ccc(S(N)(=O)=O)c(F)c2)C[C@H]1c1ccccc1 ; C=CS(=O)(=O)NC1CN(C(=O)[C@@H]2CN(C(=O)c3ccc(S(N)(=O)=O)c(F)c3)C[C@H]2c2ccccc2)C1]

and stereoisomers thereof.

In some cases, the PTGES3 inhibitor has the structure of Compound 1 or is a stereoisomers of Compound 3. For instance, Compound 3 and a stereoisomer thereof are shown below.

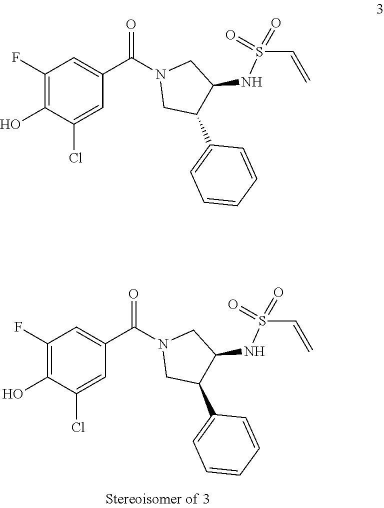 [SMILES: C=CS(=O)(=O)N[C@@H]1CN(C(=O)c2cc(F)c(O)c(Cl)c2)C[C@H]1c1ccccc1 ; C=CS(=O)(=O)N[C@@H]1CN(C(=O)c2cc(F)c(O)c(Cl)c2)C[C@@H]1c1ccccc1]

In some cases the PTGES3 inhibiting compound has a structure selected from the group consisting of:

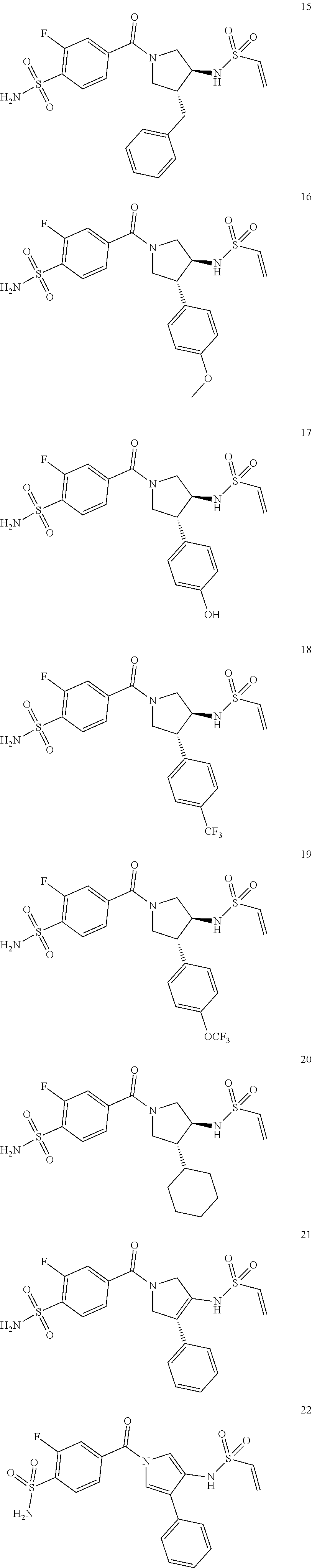

and stereoisomers thereof.

In some cases, the covalent bonds designated “x”, “y”, and “z” is are each a single bond. For example, Compound 3 shows an embodiment with such x, y, and z bonds.

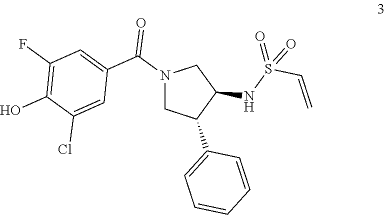 [SMILES: C=CS(=O)(=O)N[C@@H]1CN(C(=O)c2cc(F)c(O)c(Cl)c2)C[C@H]1c1ccccc1]

In some cases, the covalent bond designated “y” is a double bond and the covalent bonds designated “x” and “z” are single bonds. For example, Compound 21 shows an embodiment with such x, y, and z bonds.

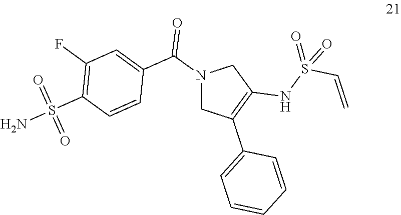 [SMILES: C=CS(=O)(=O)NC1=C(c2ccccc2)CN(C(=O)c2ccc(S(N)(=O)=O)c(F)c2)C1]

In some cases, the covalent bond designated “y” is a single bond and the covalent bonds designated “x” and “z” are double bonds. For instance, Compound 22 shows and embodiment with such x, y, and z bonds.

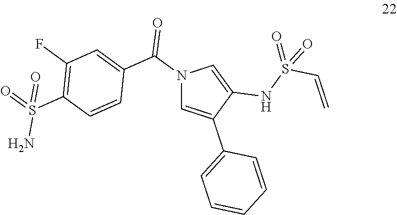 [SMILES: C=CS(=O)(=O)Nc1cn(C(=O)c2ccc(S(N)(=O)=O)c(F)c2)cc1-c1ccccc1]

### Methods

Provided are methods of treating a subject for prostate cancer.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it; (b) inhibiting the disease, i.e., arresting its development; and (c) relieving the disease, i.e., causing regression of the disease (e.g., reduction in viral titers or reducing the number or weight of cancerous cells).

In some instances, the method includes administering a PTGES3 inhibitor to the subject. In some cases, the PTGES3 inhibitor is a PTGES3 inhibitor of formula (I), e.g., a PTGES3 inhibitor of formula (Ia), formula (II), formula (IIa), formula (III), or formula (IIIa). In some embodiments, the PTGES3 inhibitor has the structure selected from the group consisting of Compounds 3-27 and stereoisomers thereof.

In some embodiments, the PTGES3 inhibitor inhibits an interaction between PTGES3 and an androgen receptor (AR). In some embodiments, the PTGES3 inhibitor is configured to form a covalent bond with a cysteine group of the androgen receptor, e.g. Cys76.

In some cases, the subject has been diagnosed with prostate cancer. As such, the method is a method inhibiting the prostate cancer or relieving the prostate cancer. For instance, the method can result in the inhibition of the prostate cancer by slowing the slowing the proliferation of the prostate cancer or by stopping the proliferation of the prostate cancer. In some cases, the method results in relieving the prostate cancer by causing regression of the prostate cancer, e.g. wherein the number of cancerous cells are reduced. In some embodiments, the method further includes diagnosing the subject with prostate cancer before the administering.

In some cases, the subject has been diagnosed with an elevated risk of prostate cancer. For instance, the method can cause a reduction in the risk of the subject developing prostate cancer. In some instances, the method includes diagnosing the subject with an elevated risk of prostate cancer. Elevated risk can be characterized as having a higher risk than a reference population, e.g. having an elevated risk compared to other people of the same age.

### Kits

Also provided are kits for use in methods of treating a subject for prostate cancer. For instance, the kit can include a PTGES3 inhibitor and packaging containing the PTGES3 inhibitor. For instance, the packaging can be a bottle, a foil sleeve, or a cardboard box. The kit can also include instructions for how the subject should administer the PTGES3 inhibitor, e.g. orally. The PTGES3 inhibitor can be part of a pharmaceutical composition, e.g. a pill or a liquid solution.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Screening for PTGES3 Interacting Compounds

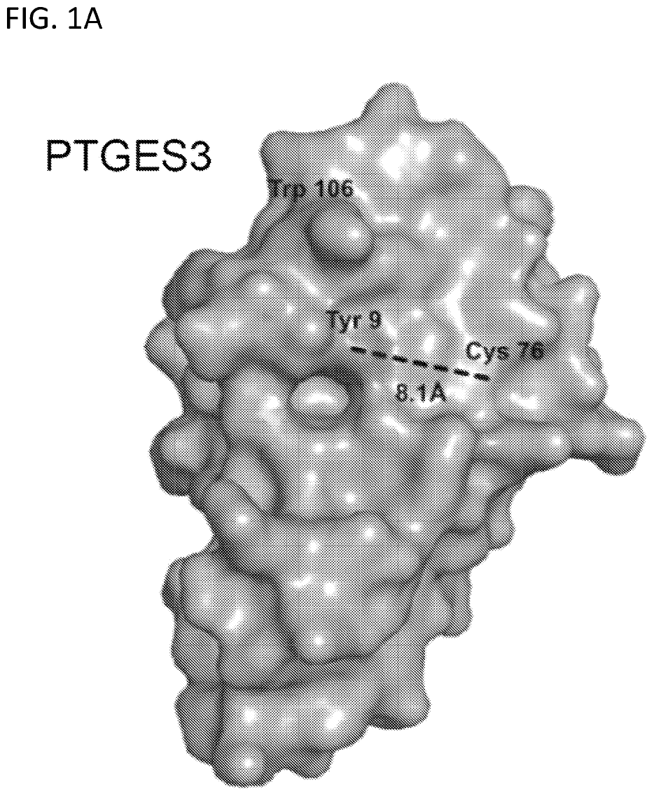

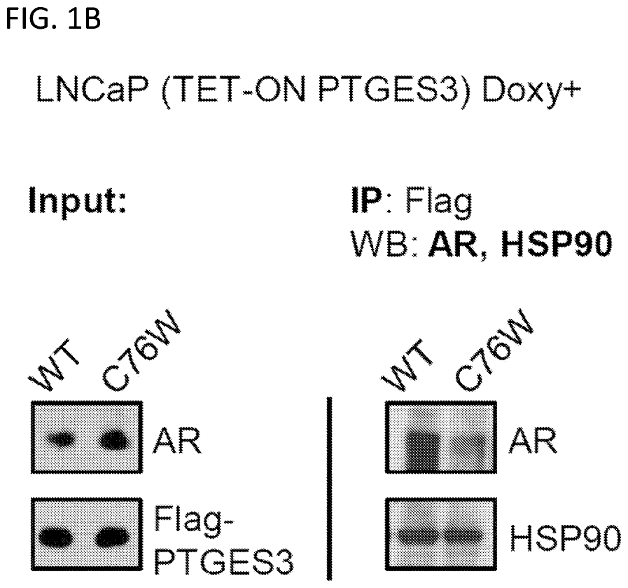

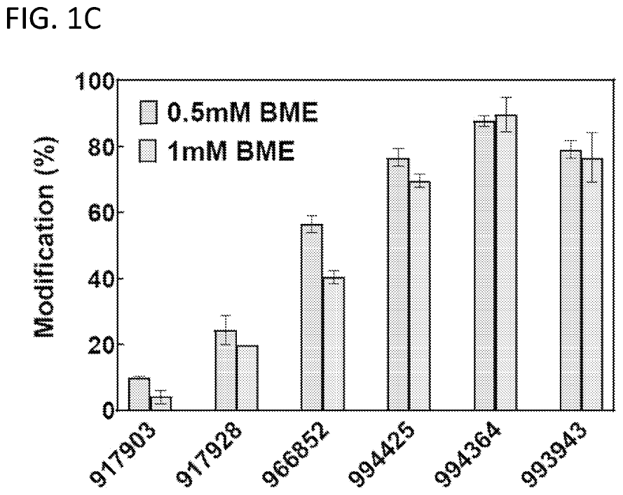

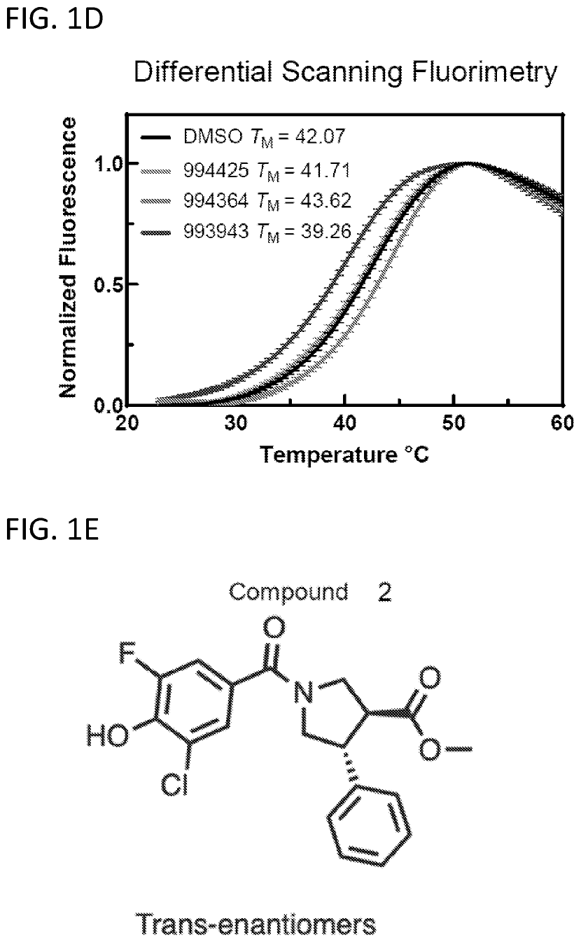

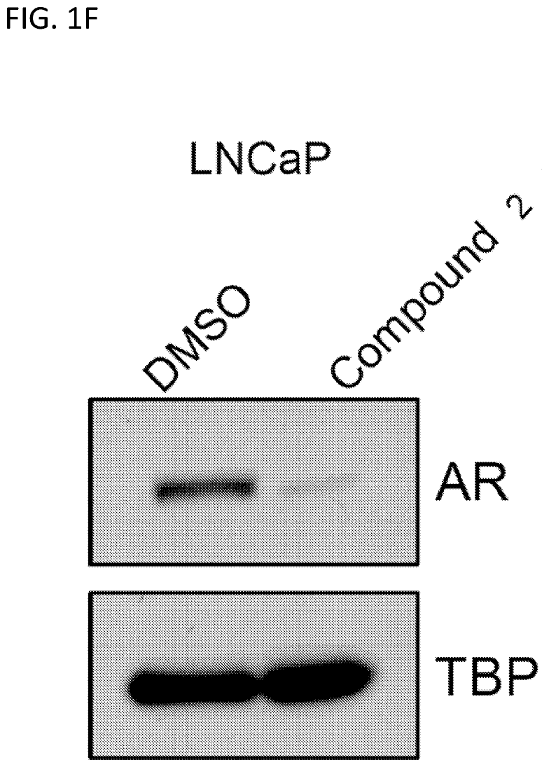

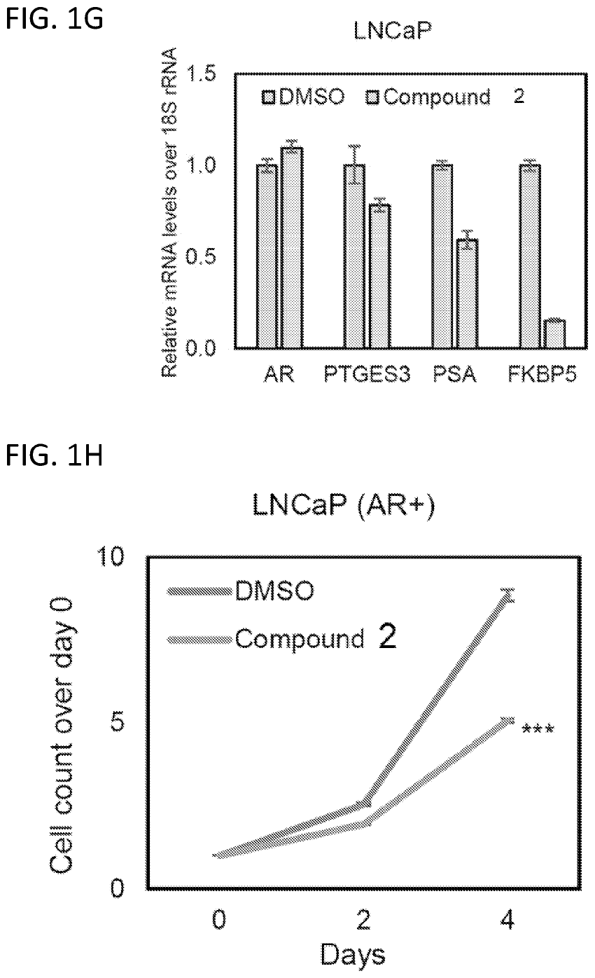

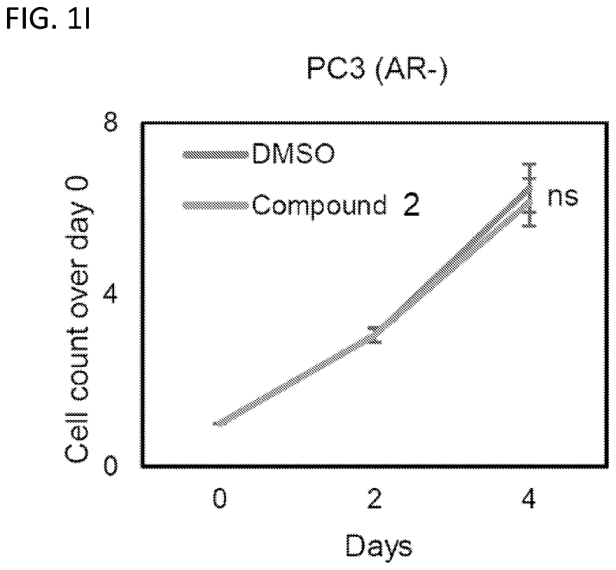

PTGES3 was evaluated and it was determined that there is a surface cysteine residue, Cys76, that is proximal to the catalytic tyrosine residue (Tyr9) and the trypotphan residue (Trp106) at the co-chaperone binding site (FIG. 1A). A study was conducted to find compounds that could bind to the Cys76 site.

First, PTES3(C76W) was expressed in LNCaP cells. The indole side chain of tryptophan can serve as a proxy for small molecule binding, and the tryptophan mutant of a protein can mimic behavior observed when a small molecule is bound (REF. 1). Co-immunoprecipitation experiments revealed that the tryptophan substitution reduced PTGES3 binding to AR, but did not affect PTGES3-HSP90 interaction. Thus, it was hypothesized that covalent modification of Cys76 by a small molecule could interrupt the PTGES3-AR interaction (FIG. 1B).

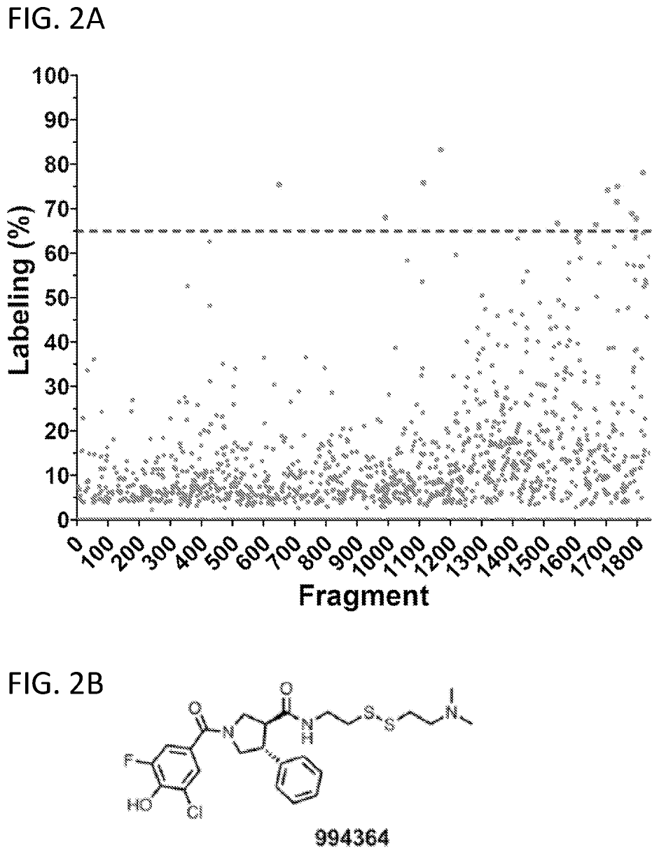

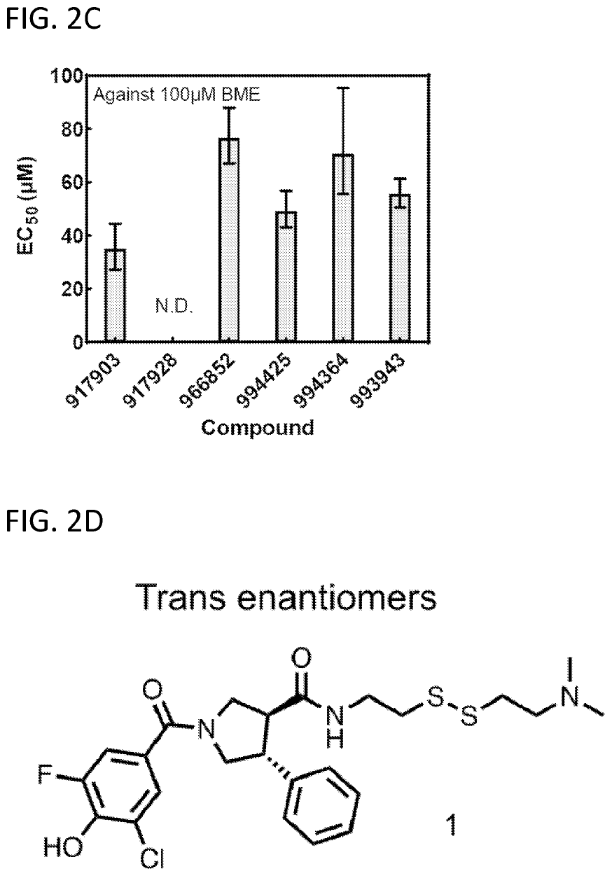

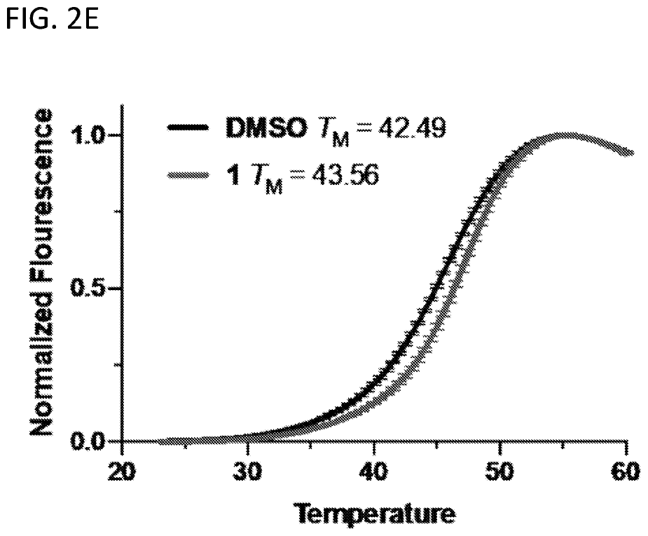

Thus, cysteine thiols reactivity was utilized in a disulfide-based fragment tethering screening platform to establish molecular scaffolds capable of binding in this critical region (REFS. 2-4). Recombinant CysLite PTGES3 was screened against a library of greater than 1800 disulfide fragments for covalent modification of Cys76 using intact protein mass spectrometry. Six hits were chosen that modified the target >65% for further evaluation (FIGS. 2A-2B).

The hits were prioritized by evaluating their EC₅₀ (concentration at which the fragment covalently modifies 50% of the protein) (FIG. 2C) and for their ability to covalently modify CysLite PTGES3 in the presence of high concentrations of competitor (FIG. 1C). The top three fragments were subsequently evaluated by differential scanning fluorimetry, and fragment hit 994364 was observed to increase the thermal melt temperature of CysLite PTGES3 (FIG. 1D).

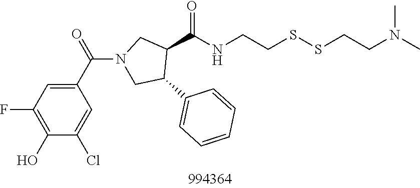 [SMILES: CN(C)CCSSCCNC(=O)[C@@H]1CN(C(=O)c2cc(F)c(O)c(Cl)c2)C[C@H]1c1ccccc1]

Fragment hit 994364 was re-synthesized and purified as a mixture of the trans-enantiomers (1) for further evaluation (FIGS. 2D-E).

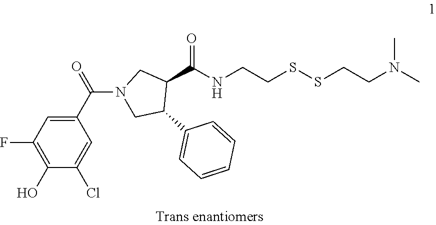 [SMILES: CN(C)CCSSCCNC(=O)[C@@H]1CN(C(=O)c2cc(F)c(O)c(Cl)c2)C[C@H]1c1ccccc1]

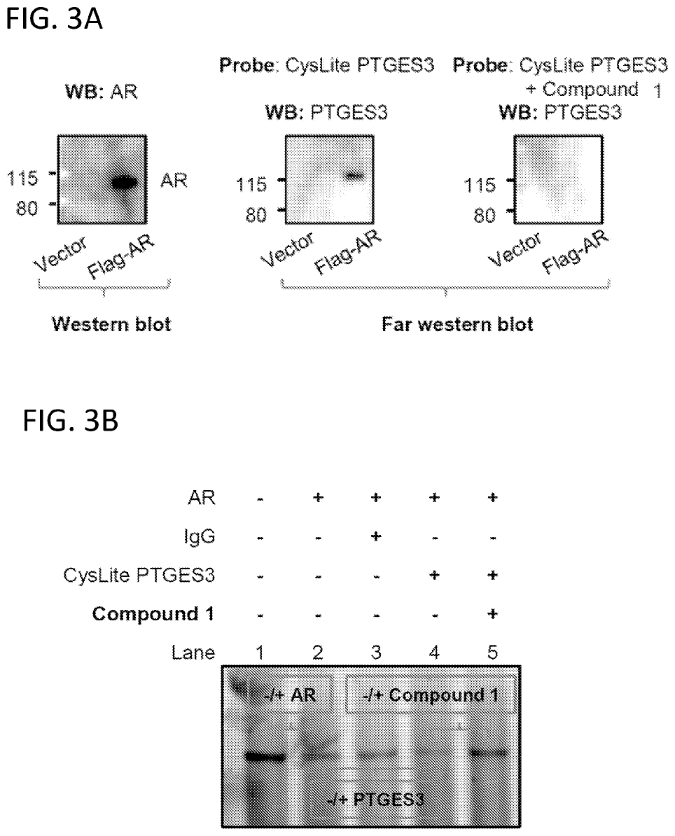

Compound 1 was first evaluated for modulation of PTGES3-AR interaction by far western blot and AR/ARE gel shift assay. Pre-modification of CysLite PTGES3 with Compound 1 ablated in vitro PTGES3-AR interaction and inhibited PTGES3 enhanced AR/ARE binding (FIGS. 3A-B).

Specificity of disulfide labeling of cysteine 76 of cyslite PTGES3 was established in the presence of BME, which acts as a non-specific competitor of cysteine labeling. Half maximal labeling of cyslite PTGES3 with compound 1 in the presence of 100 uM BME was established to be 70.59 uM. Further, 200 uM compound 1 demonstrated the ability to label cyslite PTGES3 in the presence of 500 uM and 1 mM BME to 79.03% and 76.67% respectively, demonstrating the resistance of labeling to competitor. Finally, cyslite PTGES3 fully labeled with compound 1 demonstrated an increased thermal melt temperature of 1.6° C.

| Compound 1 | |
|---|---|
| Test | Experimental Result |
| EC50 with 100 uM BME competitor | 70.59 uM |
| % Labeling in presence of 500 uM BME/1 mM | 79.03%/76.67% |
| BME competitor | |
| Thermal Shift (Cyslite PTGES3) | ΔTm + 1.6° C. |

Compound 2 was then synthesized, the non-covalent version of Compound 1 lacking the disulfide moiety, in order to evaluate cellular activity of the small molecule (FIG. 1E).

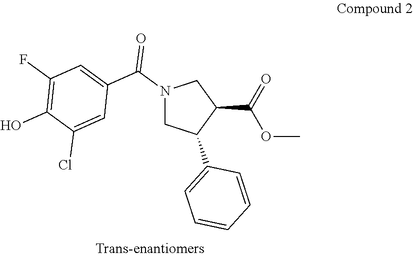 [SMILES: COC(=O)[C@@H]1CN(C(=O)c2cc(F)c(O)c(Cl)c2)C[C@H]1c1ccccc1]

Treatment of Compound 2 significantly reduced AR protein levels, the expression of AR target genes, and the growth of AR-driven LNCaP cells but not AR-independent PC3 cells (FIGS. 1F, 1H, 1I). Collectively, the small molecule binder of PTGES3 disrupts in vitro PTGES3-AR interaction and represses AR signaling in PCa cells.

Compound 2 was demonstrated to be permeable in a parallel artificial membrane permeability assay (PAMPA). Compound 2 was demonstrated to inhibit cell proliferation in AR+ but not AR− negative cell lines, suggesting an AR dependent mechanism of growth inhibition.

| Compound 2 | |
|---|---|
| Test | Experimental Result |
| PAMPA assay | Permeable |
| AR + cell line activity | Yes |

### Example 2: Additional Compounds

Based on the results with Compounds 1 and 2, additional compounds with related chemical structures were synthesized. In such compounds the disulfide group, which had been used to form a bond with the cysteine residue of PTGES3, was replaced with a vinyl sulfonamide group.

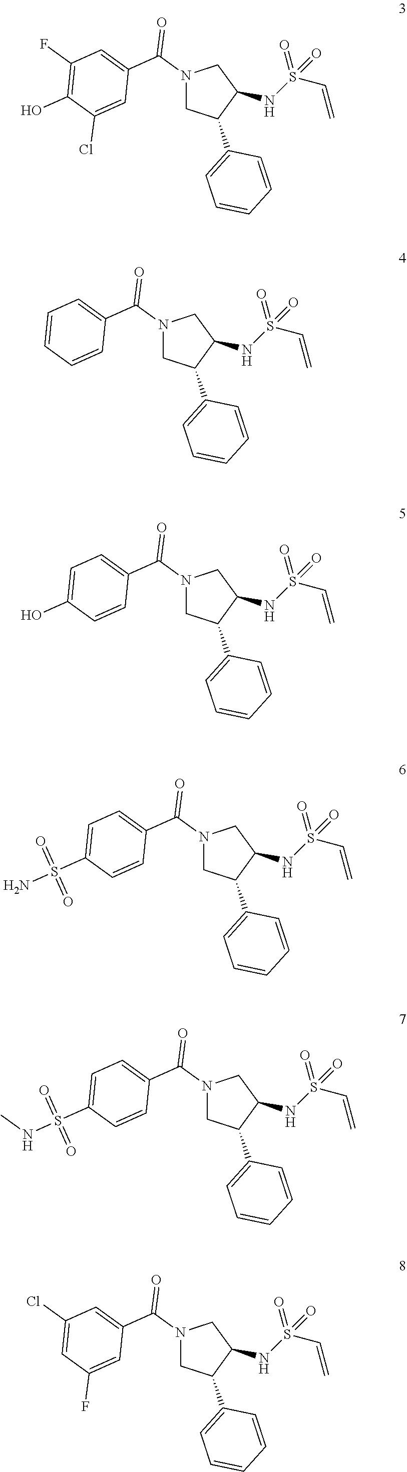

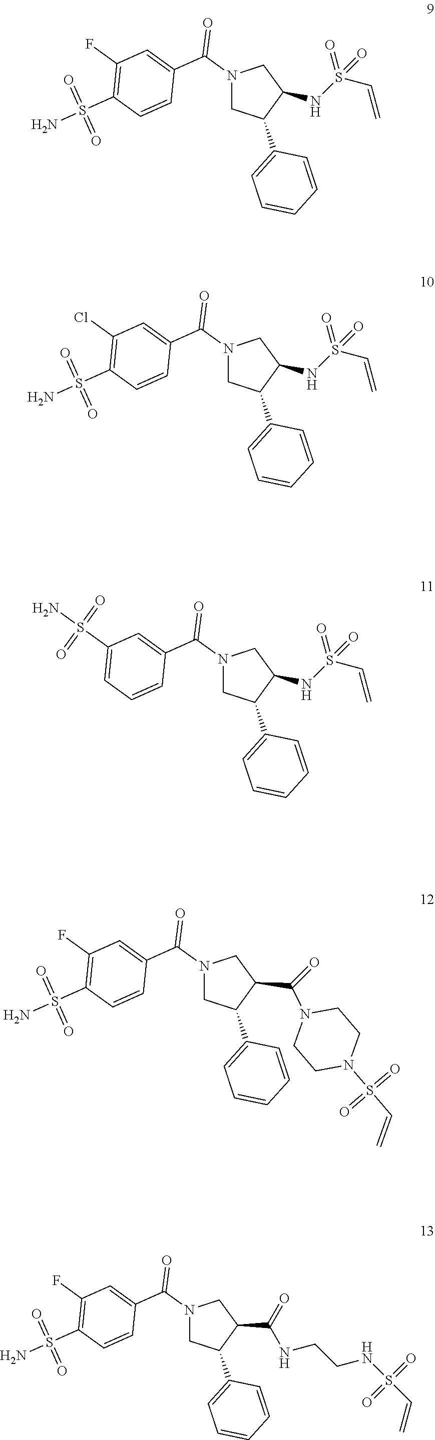 [SMILES: C=CS(=O)(=O)N[C@@H]1CN(C(=O)c2ccc(S(N)(=O)=O)c(F)c2)C[C@H]1c1ccccc1 ; C=CS(=O)(=O)N[C@@H]1CN(C(=O)c2ccc(S(N)(=O)=O)c(Cl)c2)C[C@H]1c1ccccc1 ; C=CS(=O)(=O)N[C@@H]1CN(C(=O)c2cccc(S(N)(=O)=O)c2)C[C@H]1c1ccccc1 ; C=CS(=O)(=O)N1CCN(C(=O)[C@@H]2CN(C(=O)c3ccc(S(N)(=O)=O)c(F)c3)C[C@H]2c2ccccc2)CC1 ; C=CS(=O)(=O)NCCNC(=O)[C@@H]1CN(C(=O)c2ccc(S(N)(=O)=O)c(F)c2)C[C@H]1c1ccccc1]

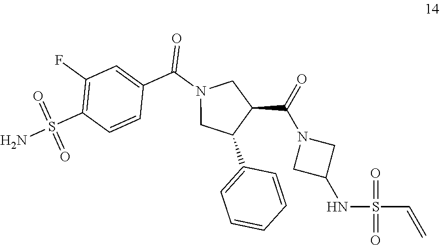 [SMILES: C=CS(=O)(=O)NC1CN(C(=O)[C@@H]2CN(C(=O)c3ccc(S(N)(=O)=O)c(F)c3)C[C@H]2c2ccccc2)C1]

Compounds were tested at 100 uM for the ability to label cysteine 76 of cyslite PTGES3. In some cases labeling was not detected. Cyslite PTGES3 was then fully labeled with the compounds and the thermal shift values of the fully labeled protein was determined with stabilization values indicated below. In some cases, compounds did not fully label cyslite PTGES3 and stabilization was not determined. Finally, select compounds were tested to determine if they inhibited cell growth in AR+LNCaP cells.

| | 4 h | 24 h | Thermal Shift | |
|---|---|---|---|---|
| | labeling | labeling | (Cyslite | |
| Compound | (100 uM) | (100 uM) | PTGES3) | Cell activity |
| 3 | 5.27% | 28.15% | ΔTm + 3.3° C. | |
| 4 | 4.01% | 20.75% | ΔTm + 1.9° C. | |
| 5 | 4.71% | 35.51% | Not determined | |
| 6 | 9.61% | 44.20% | ΔTm + 3.0° C. | Yes |
| 7 | 7.21% | 36.40% | ΔTm + 2.1 C. | |
| 8 | Not detected | 14.74% | Not determined | |
| 9 | 12.38% | 51.4% | ΔTm + 3.3° C. | Yes |
| 10 | Not detected | 27.44% | ΔTm + 3.3° C. | |
| 11 | 17.84 | 72.62 | ΔTm + 0.4° C. | Yes |
| 12 | 6.27% | 36.65% | No stabilization | |
| 13 | Not detected | 13.63% | No stabilization | |
| 14 | 7.18% | 48.46% | No stabilization | No |

### Methods

### Cell Lines

LNCaP cell lines were grown in the RPMI 1640 medium (Gibco) supplemented with 10% fetal bovine serum (FBS; Gibco). Cells were grown in a humidified 5% CO₂ incubator at 37° C.

### Tethering Screen

Recombinant CysLite PTGES3 (residues 1-125) at 1 μM was reacted with a 200 μM fragment and 100 μM PME in 50 mM Tris-HCl, pH 8.0, 150 mM NaCl for 1 hour at ambient temperature. Reactions were quenched by the addition of formic acid to a final concentration of 0.4%. The extent of modification was assessed by electrospray mass spectrometry using a Waters Acquity UPLC/ESI-TQD. By setting a threshold of >55% modification, we achieved a hit rate of 1.7%.

### Dose Response Assay

Recombinant CysLite PTGES3 (1 μM, 50 mM Tris-HCl, pH 8.0, 150 mM NaCl and 100 μM βME) were incubated for 1 h at ambient temperature with lead fragments (4% DMSO) in a 7-, 8- or 9-point threefold serial dilution series in duplicate. Reactions were quenched by the addition of formic acid to a final concentration of 0.4%. Labeling was determined by intact protein mass spectrometry.

### BME Competition Assay

Recombinant CysLite PTGES3 (1 μM, 50 mM Tris-HCl, pH 8.0, 150 mM NaCl) were incubated with 200 μM fragment (2% DMSO) and 500 μM or 1 mM BME at ambient temperature for 8 h. Reactions were quenched by the addition of formic acid to a final concentration of 0.4%. Labeling was determined by intact protein mass spectrometry.

### Differential Scanning Fluorimetry

Recombinant CysLite PTGES3 at a concentration of 8 μM in 50 mM HEPES pH 7.5, 150 mM NaCl were incubated with compounds or DMSO for unlabeled comparisons, at ambient temperature until complete labeling was observed by intact protein mass spectrometry. 7K Zeba Desalting columns (ThermoFisher) were used to remove excess compound. The Zeba 7K columns were first preequilibrated with 50 mM HEPES (pH 7.5), 150 mM NaCl. Sypro Orange (500x, DMSO) was added to the purified protein-small molecule conjugates to a final concentration of 5x Sypro Orange. These DSF assays were performed in triplicate in white 96-well plates. The plate was sealed with optically clear PCR sealing film (USA Scientific). The thermal shift assay was performed on a Bio-Rad CFX qPCR instrument. The temperature was increased from 23° C. to 95° C. at a rate of 0.5° C./s. After each temperature step, fluorescence was monitored with an excitation of 492 nm and emission of 610 nm. Raw fluorescence values were normalized between 0 and 1 and fit to a Boltzman equation using GraphPad Prism with the maximum value of the fit constrained to 1.

### EMSA (Gel Shift) Assays

Electrophoretic mobility shift assays (EMSA) were performed using recombinant AR protein (EMD Millipore) and 3×ARE dsDNA from the ARR3TK plasmid. Protein-DNA complexes were allowed to incubate in room temperature for 30 min in loading buffer (20 mM HEPES, 100 mM KCl, 2 ng/ml poly(dI-dC), 10% glycerol, and 20 nM DHT), followed by electrophoresis on 6% DNA Retardation Gels in TBE buffer (PH 8.0). Visualization of protein-DNA complexes was performed with SYBR Gold DNA staining dye (Invitrogen).

### Statistical Analysis

Spearman's correlation was used to determine statistical significance for all the correlation plots. For gene expression and correlation, the Wilcoxon rank-sum test was used to test for differences between two groups, unless otherwise stated. Unpaired t-test were used to determine statistical analysis for the column plots, denoted by asterisk (*). *p<0.05, **p<0.01, ***p<0.001. Two-way ANOVA was used to determine statistical significance in the in vivo data. In RNA-Seq data, the Benjamini-Hochberg test was performed. Corrected p-value <0.05 and log 2 foldchange >0.5 or <0.5 were considered statistically significant. In ATAC-Seq data peak regions with Benjamini-Hochberg corrected p-value <0.05 and log 2 foldchange >0.5 or <0.5 were considered statistically significant.

### REFERENCES

1. Taylor, I. R. et al. Tryptophan scanning mutagenesis as a way to mimic the compound-bound state and probe the selectivity of allosteric inhibitors in cells. Chem. Sci. 11, 1892-1904 (2020).
2. Erlanson, D. A. et al. Site-directed ligand discovery. Proc. Natl. Acad Sci. U.S.A. 97, 9367-9372 (2000).
3. Ostrem, J. M., Peters, U., Sos, M. L., Wells, J. A. & Shokat, K. M. K-Ras(G12C) inhibitors allosterically control GTP affinity and effector interactions. Nature 503, 548-551 (2013).
4. Hallenbeck, K. K. et al. A Liquid Chromatography/Mass Spectrometry Method for Screening Disulfide Tethering Fragments. SLAS Discov. Adv. Sci. Drug Discov. 23, 183-192 (2018).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) is not invoked.

What is claimed is:

1. A prostaglandin E synthase 3 (PTGES3) inhibitor compound of formula (I): 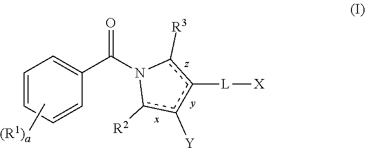
wherein:
each R¹, R² and R³ is independently selected from H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof;
L is a linking group or absent;
X is a thiol covalently bonding moiety;
Y is —(CH₂)ₙR⁵, wherein n is 0 or 1, wherein R⁵ is selected from aryl, substituted aryl, heteroaryl, substituted heteroaryl, cycloalkyl, and substituted cycloalkyl;
a is an integer ranging from 0 to 5; and
the covalent bonds designated “x”, “y” and “z” are each independently a single bond or a double bond provided that if bond “y” is a double bond then bonds “x” and “z” are single bonds.

2. The compound of claim 1, wherein the compound has formula (Ia):

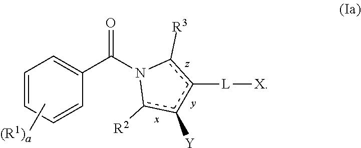

3. The compound of any one of claims 1-2, wherein a ranges from 1 to 5 and at least one R¹ is selected from halo, hydroxy, sulfonylamine.

4. The compound of any one of claims 1-3, wherein the thiol covalently bonding moiety is an electrophilic group.

5. The compound of any one of claims 1-4, wherein the thiol covalently bonding moiety is selected from the group consisting of a vinyl sulfone, a maleimide, a α-halocarbonyl, an acrylamide, an iodoacetamide, α-haloamide, an epoxide, an azirdine, and a β-haloethylamine.

6. The compound of claim 5, wherein the thiol covalently bonding moiety is a vinyl sulfone moiety.

7. The compound of claim 6, wherein the vinyl sulfone moiety is a vinyl sulfonamide moiety.

8. The compound of any one of claims 1-7, wherein L is absent.

9. The compound of any one of claims 1-7, wherein L is a linking group selected from the group consisting of alkylene, alkenylene, alkynylene, cycloalkylene, heterocyclylene, arylene, heteroarylene, amino, acylene, and substituted versions thereof.

10. The compound of any one of claims 1-9, wherein the compound has formula (II): 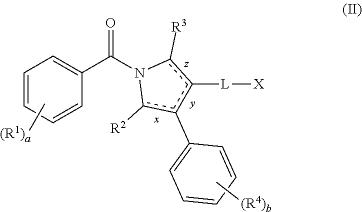
wherein:
each R⁴ is independently selected from alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and
b is an integer ranging from 0 to 5.

11. The compound of claim 10, wherein the compound has formula (IIa):

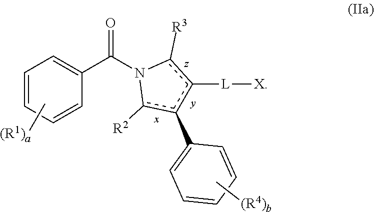

12. The compound of any one of claims 1-11, wherein the compound has formula (III): 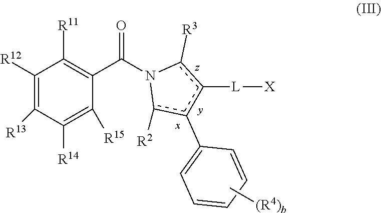
wherein:
R¹¹, R¹, R¹³, R¹⁴, and R¹⁵ are each independently selected from H, halo, hydroxy, and sulfonylamine.

13. The compound of claim 12, wherein the compound has formula (IIIa):

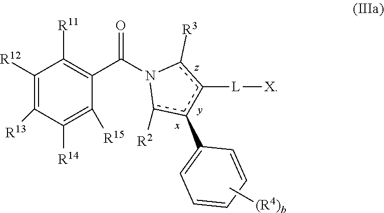

14. The compound of any one of claims 12-13, wherein R¹¹ is H.

15. The compound of any one of claims 12-14, wherein R¹² is halo.

16. The compound of claim 15, wherein R¹² is fluoro.

17. The compound of any one of claims 12-16, wherein R¹³ is hydroxy.

18. The compound of any one of claims 12-16, wherein R¹³ is sulfonylamine.

19. The compound of any one of claims 12-18, wherein R¹⁴ is halo.

20. The compound of claim 19, wherein R¹⁴ is chloro.

21. The compound of any one of claims 12-20, wherein R¹⁵ is H.

22. The compound of any one of claims 12-21, wherein R² is H.

23. The compound of any one of claims 12-22, wherein R³ is H.

24. The compound of any one of claims 1-23, wherein the covalent bonds designated “x”, “y”, and “z” are each a single bond.

25. The compound of any one of claims 1-23, wherein the covalent bond designated “y” is a double bond and the covalent bonds designated “x” and “z” are single bonds.

26. The compound of any one of claims 1-23, wherein the covalent bond designated “y” is a single bond and the covalent bonds designated “x” and “z” are double bonds.

27. The compound of claim 1, wherein the compound has a structure selected from the group consisting of: 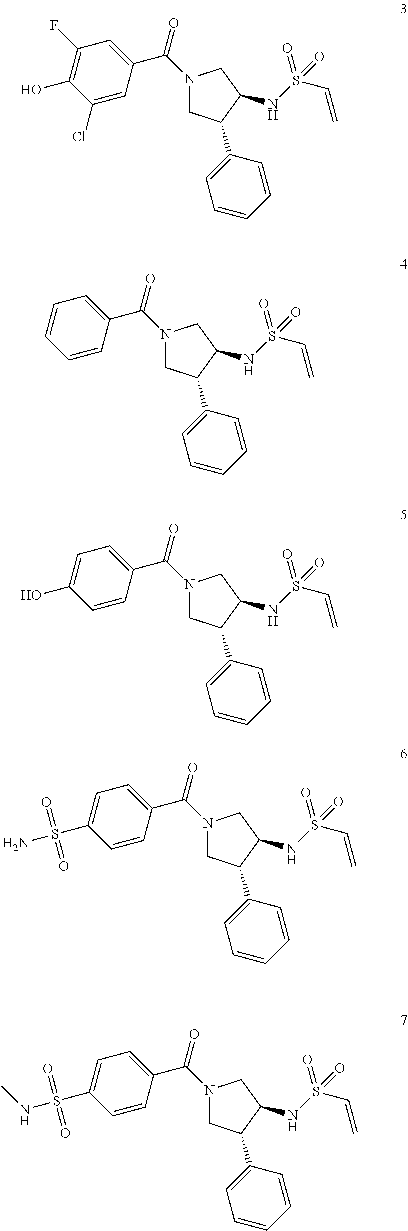 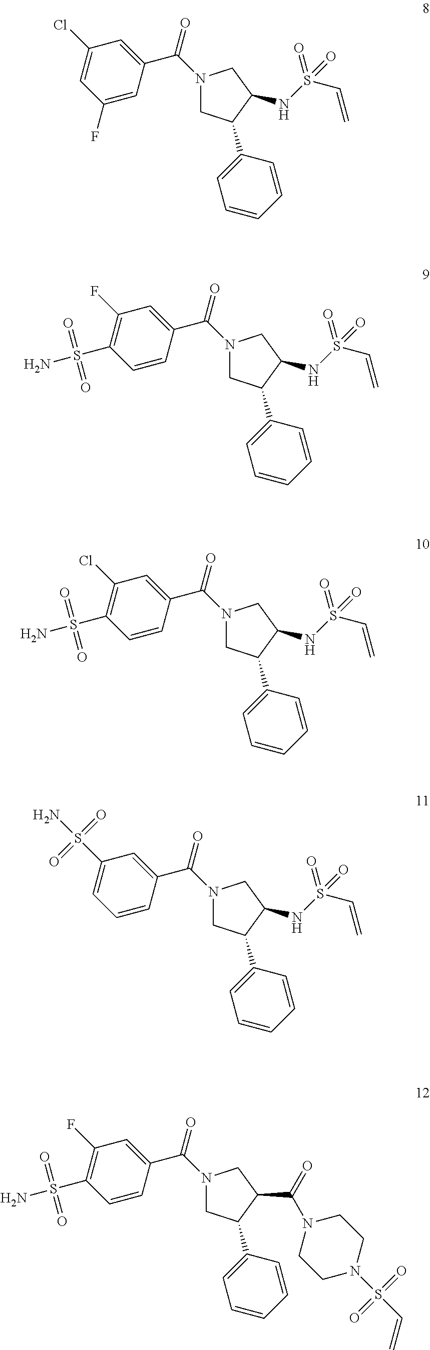 [SMILES: C=CS(=O)(=O)N[C@@H]1CN(C(=O)c2cc(F)cc(Cl)c2)C[C@H]1c1ccccc1 ; C=CS(=O)(=O)N[C@@H]1CN(C(=O)c2ccc(S(N)(=O)=O)c(F)c2)C[C@H]1c1ccccc1 ; C=CS(=O)(=O)N[C@@H]1CN(C(=O)c2ccc(S(N)(=O)=O)c(Cl)c2)C[C@H]1c1ccccc1 ; C=CS(=O)(=O)N[C@@H]1CN(C(=O)c2cccc(S(N)(=O)=O)c2)C[C@H]1c1ccccc1 ; C=CS(=O)(=O)N1CCN(C(=O)[C@@H]2CN(C(=O)c3ccc(S(N)(=O)=O)c(F)c3)C[C@H]2c2ccccc2)CC1] 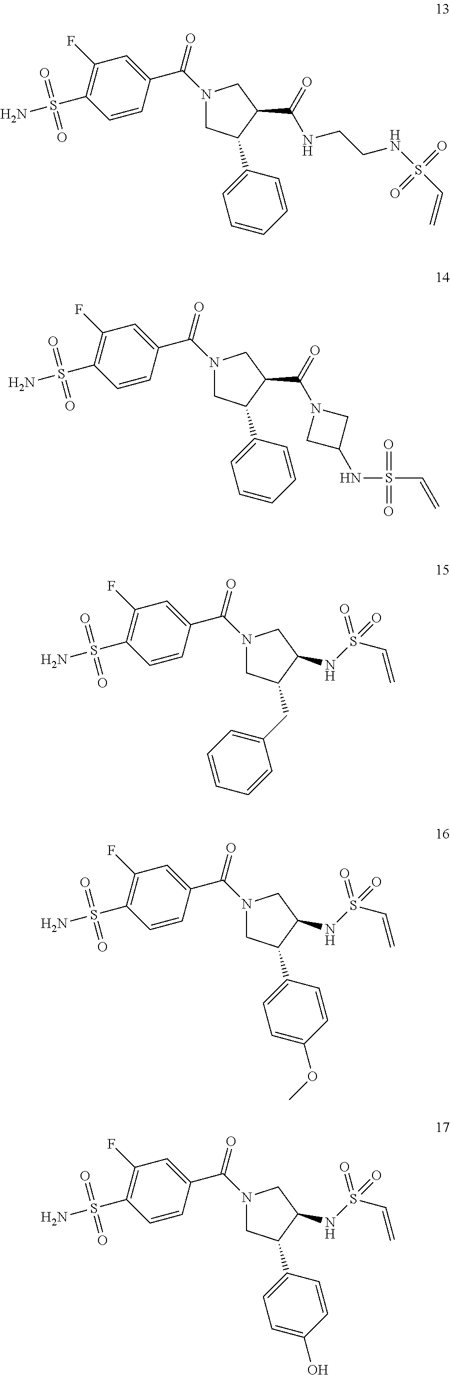 [SMILES: C=CS(=O)(=O)NCCNC(=O)[C@@H]1CN(C(=O)c2ccc(S(N)(=O)=O)c(F)c2)C[C@H]1c1ccccc1 ; C=CS(=O)(=O)NC1CN(C(=O)[C@@H]2CN(C(=O)c3ccc(S(N)(=O)=O)c(F)c3)C[C@H]2c2ccccc2)C1 ; C=CS(=O)(=O)N[C@@H]1CN(C(=O)c2ccc(S(N)(=O)=O)c(F)c2)C[C@H]1Cc1ccccc1 ; C=CS(=O)(=O)N[C@@H]1CN(C(=O)c2ccc(S(N)(=O)=O)c(F)c2)C[C@H]1c1ccc(OC)cc1 ; C=CS(=O)(=O)N[C@@H]1CN(C(=O)c2ccc(S(N)(=O)=O)c(F)c2)C[C@H]1c1ccc(O)cc1] 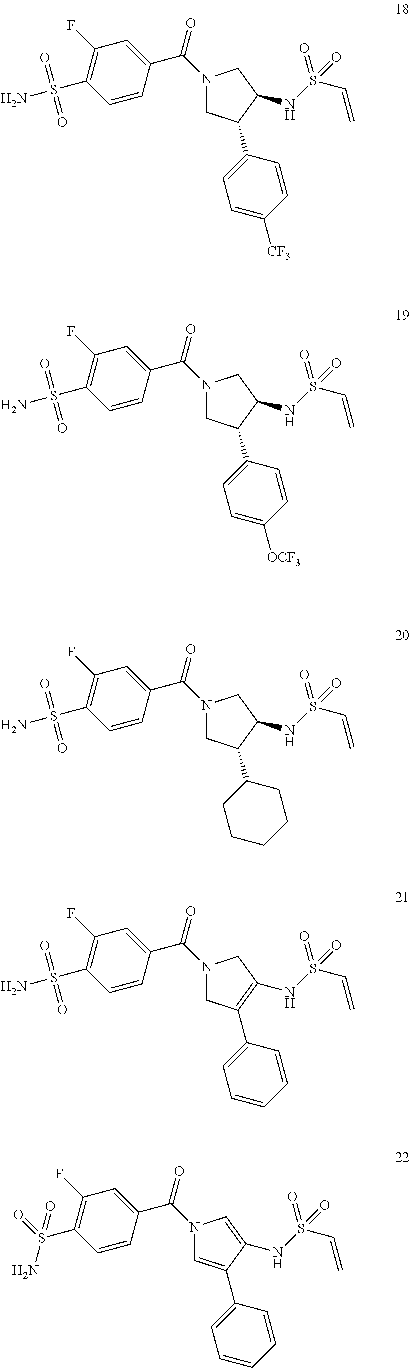 [SMILES: C=CS(=O)(=O)N[C@@H]1CN(C(=O)c2ccc(S(N)(=O)=O)c(F)c2)C[C@H]1c1ccc(C(F)(F)F)cc1 ; C=CS(=O)(=O)N[C@@H]1CN(C(=O)c2ccc(S(N)(=O)=O)c(F)c2)C[C@H]1c1ccc(OC(F)(F)F)cc1 ; C=CS(=O)(=O)N[C@@H]1CN(C(=O)c2ccc(S(N)(=O)=O)c(F)c2)C[C@H]1C1CCCCC1 ; C=CS(=O)(=O)NC1=C(c2ccccc2)CN(C(=O)c2ccc(S(N)(=O)=O)c(F)c2)C1 ; C=CS(=O)(=O)Nc1cn(C(=O)c2ccc(S(N)(=O)=O)c(F)c2)cc1-c1ccccc1]
and stereoisomers thereof.

28. A method of treating a subject for prostate cancer, the method comprising:
administering a prostaglandin E synthase 3 (PTGES3) inhibitor to the subject.

29. The method of claim 28, wherein the PTGES3 inhibitor inhibits an interaction between PTGES3 and an androgen receptor (AR).

30. The method of any one of claims 28-29, wherein the PTGES3 inhibitor is configured to form a covalent bond with a cysteine group of the androgen receptor.

31. The method of claim 30, wherein the cysteine group of the androgen receptor is Cys76.

32. The method of any one of claims 28-31, wherein the PTGES3 inhibitor is a PTGES3 inhibitor of any one of claims 1-27.

33. The method of any one of claims 28-32, wherein the subject has been diagnosed with prostate cancer.

34. The method of claim 33, further comprising diagnosing the subject with prostate cancer.

35. The method of any one of claims 38-32, wherein the subject has been diagnosed with an elevated risk of prostate cancer.

36. The method of claim 35, further comprising diagnosing the subject with an elevated risk of prostate cancer.

37. A kit for treating a subject for prostate cancer, the kit comprising:
a prostaglandin E synthase 3 (PTGES3) inhibitor of any one of claims 1-27; and
packaging containing the PTGES3 inhibitor.
